(19) United States
(12) Patent Application Publication
(10) Pub. No.: US 2015/0299286 A1
(43) Pub. Date: Oct. 22, 2015

(54) GLUTAMIC ACID-STABILIZED INSULIN ANALOGUES

(71) Applicant: CASE WESTERN RESERVE UNIVERSITY, Cleveland, OH (US)

(72) Inventor: Michael A. Weiss, Moreland Hills, OH (US)

(21) Appl. No.: 14/373,621

(22) PCT Filed: Jan. 22, 2013
(86) PCT No.: PCT/US13/22551

§ 371 Date: Jul. 21, 2014

Related U.S. Application Data
(60) Provisional application No. 61/589,271, filed on Jan. 20, 2012.

(51) Int. Cl. C07K 14/62

(52) U.S. Cl. CPC C07K 14/62; A61K 38/00

(57) ABSTRACT

An insulin analogue comprises a B-chain polypeptide containing the acidic two-residue extension GluB31-GluB32, and optionally an A-chain polypeptide containing acidic substitution GluA8, and additionally optionally a non-standard modification of PheB24. The analogue may also contain additional B-chain substitutions known in the art to enhance the rate of absorption of an insulin analogue formulation following subcutaneous injection or infusion. The analogue may be an analogue of a mammalian insulin, such as human insulin. A nucleic acid encoding such an insulin analogue is also provided. A method of treating a patient comprises administering a physiologically effective amount of the insulin analogue or a physiologically acceptable salt thereof to a patient. The analogue may be administered at a high concentration while maintaining fast-acting properties. A method of semi-synthesis using an unprotected octapeptide by means of modification of an endogenous tryptic site by non-standard amino-acid substitutions.

### STATEMENT REGARDING FEDERALLY SPONSORED RESEARCH OR DEVELOPMENT

This invention was made with government support under cooperative agreements awarded by the National Institutes of Health under grant numbers DK40949 and DK074176. The U.S. government may have certain rights to the invention.

### BACKGROUND OF THE INVENTION

This invention relates to polypeptide hormone analogues that exhibits enhanced pharmaceutical properties, such as greater thermodynamic stability, greater biological activity, or more rapid pharmacokinetics at polypeptide concentrations greater than are ordinarily employed in pharmaceutical formulations. The present invention pertains to insulin, a polypeptide hormone of two chains that regulates vertebrate metabolism and is widely used in humans and other mammals for the treatment of diabetes mellitus. The sequence of insulin is shown in schematic form in FIG. 1; individual residues are indicated by the identity of the amino acid (typically using a standard three-letter code), the chain and sequence position (typically as a superscript).

The three Glutamic acid residues provided at positions A8, B31, and B32 increase the net negative charge of the insulin molecule and of the zinc-stabilized hexamer thereof when dissolved in a solution whose pH is in the range 6.5-8.0 as is desirable in a pharmaceutical formulation. This invention enables the formulation of insulin analogs at concentrations higher than 100 units per ml (U-100) such that (i) the thermodynamic stability of the insulin analogue is similar to or greater than that of wild-type insulin, (ii) biological potency is similar to or greater than that of wild-type insulin, (iii) rapid-acting pharmacokinetic (PK) and pharmacodynamic (PD) properties are retained relative to wild-type human insulin at a U-100 concentration and such that (iv) their mitogenic properties are similar to wild-type human insulin or insulin analogues in current clinical use.

The engineering of non-standard proteins, including therapeutic agents and vaccines, may have broad medical and societal benefits. An example of a medical benefit would be optimization of the pharmacokinetic properties of a protein. An example of a further societal benefit would be the engineering of proteins amenable to formulation at high protein concentrations with deterioration of the PK/PD properties of the formulation. A further example of a society benefit is a protein formulation with prolonged shelf life. An example of a therapeutic protein is provided by insulin. Analogues of insulin containing a greater net negative charge at neutral pH and optionally non-standard amino-acid substitutions may in principle exhibit superior properties with respect to stability, biological potency, or PK/PD or the dependence of PK/PD on the concentration of insulin in the formulation. The latter is of particular importance in public health as highly concentrated formulations of insulin may bring medical benefits to patients with obesity and marked insulin resistance; such patients are often African-American women and other disadvantaged minorities. The challenge posed by the pharmacokinetics of insulin absorption following subcutaneous injection affects the ability of patients with diabetes mellitus (DM) to achieve tight glycemic control and constrains the safety and performance of insulin pumps.

Insulin resistance is a condition in which the classical target tissues of this hormone (adipose tissue, muscle, and liver) require a higher concentration of insulin or insulin analogue in the blood stream to achieve the same biological response as healthy subjects exhibit in response to normal concentrations of insulin in the blood stream. Insulin resistance commonly accompanies Type 2 diabetes mellitus. A particular medical need is posed by the marked resistance to insulin exhibited by certain patients with DM associated with obesity, by certain patients with DM associated with a genetic predisposition to insulin resistance, and by patients with DM secondary to lipodystrophy, treatment with corticosteroids, or over-secretion of endogenous corticosteroids (Cushing's Syndrome). The number of patients with marked insulin resistance is growing due to the obesity pandemic in the developed and developing worlds (leading to the syndrome of “diabesity”) and due to the increasing recognition of a monogenic form of DM arising from a mutation in mitochondrial DNA in which insulin resistance can be unusually severe (van den Ouweland, J. M., Lemkes, H. H., Ruitenbeek, W., Sandkuijl, L. A., de Vijlder, M. F., Struyvenberg, P. A., van de Kamp, J. J., & Maassen, J. A. (1992) Mutation in mitochondrial tRNA(Leu)(UUR) gene in a large pedigree with maternally transmitted type II diabetes mellitus and deafness. Nature Genet. 1, 368-71). Treatment of such otherwise diverse subsets of patients typically requires the subcutaneous injection of large volumes of regular insulin formulations (U-100 strength; ordinarily 0.6 mM insulin or insulin analogue). Injection of such large volumes can lead to pain and variability in the rate of onset and duration of insulin action. Although a U-500 formulation of wild-type insulin is available for clinical use (sold under the trademark Humulin® R U-500; Eli Lilly and Co.), the increase in insulin concentration from 0.6 mM to 3 mM leads to a delay in the onset, and prolongation, of insulin action such that the PK/PD properties of Humulin® R U-500 (or similar such product) resemble those of a micro-crystalline suspension of protamine-zinc-containing insulin hexamers; this formulation has long been designated neutral protamine Hagadorn (NPH). Prandial use of a U-500 formulation of wild-type insulin by subcutaneous injection would thus be expected to decrease the efficacy of glycemic control and increase the risk of hypoglycemic episodes. Use of Humulin® R U-500 (or similar such product) in a device for continuous subcutaneous insulin infusion (CSII; an “insulin pump”) would likewise be expected to interfere with the ability of the patient or control algorithm to make effective adjustments in insulin infusion rates based on current or past measurements of blood glucose concentrations, leading to suboptimal glycemic control and increased risk of hypoglycemic events.

A well-established principle of insulin pharmacology relates the aggregation state of the injected insulin molecule to the time course of absorption from the depot into capillaries and hence into the systemic circulation. In general the more aggregated are the insulin molecules into high-molecular weight complexes, the greater the delay in absorption and more prolonged the insulin action. Amino-acid substitutions in the insulin molecule that weaken its self-assembly are known in the art to be associated with more rapid absorption relative to wild-type human insulin; examples are provided by the substitution Pro^{B28}→Asp (insulin aspart, the active component of the insulin product sold under the trademark Novolog®; Novo-Nordisk, Ltd) and by the paired substitutions Pro^{B28}→Lys and Lys^{B29}→Pro (insulin Lispro, the active component of the insulin product sold under the trademark Humalog®; Eli Lilly and Co.). Conversely, amino-acid extensions or chemical modifications of the insulin molecule that cause a shift in its isoelectric point (pI) from ca. pH 5 to ca. pH 7 are known in the art to lead to isoelectric precipitation of the modified insulin in the subcutaneous depot; such high molecular-weight complexes provide prolonged absorption as a basal insulin formulation. Examples are provided by the insulin product sold under the trademark NovoSol Basal® (a discontinued product of Novo-Nordisk in which Thr^{B27} was substituted by Arg and in which the C-terminal carboxylate moiety of Thr^{B30} was amidated) and insulin glargine (the active component of the insulin product sold under the trademark Lantus®, a basal formulation in which the B chain was extended by the dipeptide Arg^{B31}-Arg^{B32}; Sanofi-Aventis, Ltd.). (NovoSol Basal® and Lantus® each contain the additional substitution Asn^{A21}→Gly to enable their soluble formulation under acidic conditions (pH 3 and pH 4 respectively) without chemical degradation due to deamidation of Asn^{A21}.) Prolongation of classical micro-crystalline insulin suspensions (NPH, semi-lente, lente, and ultra-lente) exhibit a range of intermediate-to-long-acting PK/PD properties reflecting the respective physico-chemical properties of these micro-crystals and their relative rates of dissolution.

The above insulin products, including current and past formulations of wild-type human insulin or animal insulins, employ or employed self-assembly of the insulin molecule as a means to achieve chemical stability, as a means to avoid fibril formation, as a means to modulate PK/PD properties, or as a means to achieve a combination of these objectives. Yet insulin self-assembly can also introduce unfavorable or undesired properties. The non-optimal prolonged PK/PD properties of Humulin® R U-500 (or a similar such product), for example, are likely to be the result of hexamer-hexamer associations in the formulation and in the subcutaneous depot (FIG. 2). Indeed, studies of wild-type bovine insulin zinc hexamers in vitro by laser light scattering have provided evidence of progressive hexamer-hexamer interactions in the concentration range 0.3-3 mM. Current and past strategies for the composition of insulin formulations and design of insulin analogues therefore face and have faced an irreconcilable barrier to the development of a rapid-acting ultra-concentrated insulin formulation: whereas self-assembly is necessary to obtain chemical and physical stability, its progressive nature above 0.6 mM leads to unfavorable prolongation of PK/PD. Irrespective of theory, we therefore sought to invent an insulin analogue with PK/PD properties similar to or more rapid than regulation formulations of wild-type human insulin at U-100 strength (e.g., Humulin R® U-100; Eli Lilly and Co.) such that these PK/PD properties are not significantly affected by the concentration of insulin analogue in the range 0.6 mM-3.0 mM.

Administration of insulin has long been established as a treatment for diabetes mellitus. Insulin is a small globular protein that plays a central role in metabolism in vertebrates. Insulin contains two chains, an A chain, containing 21 residues, and a B chain containing 30 residues. The hormone is stored in the pancreatic β-cell as a Zn²⁺-stabilized hexamer, but functions as a Zn²⁺-free monomer in the bloodstream. Insulin is the product of a single-chain precursor, proinsulin, in which a connecting region (35 residues) links the C-terminal residue of B chain (residue B30) to the N-terminal residue of the A chain. Crystalline arrays of zinc insulin hexamers within mature storage granules have been visualized by electron microscopy (EM).

A major goal of insulin replacement therapy in patients with DM is tight control of the blood glucose concentration to prevent its excursion above or below the normal range characteristic of healthy human subjects. Excursions below the normal range are associated with immediate adrenergic or neuroglycopenic symptoms, which in severe episodes lead to convulsions, coma, and death. Excursions above the normal range are associated with increased long-term risk of microvascular disease, including retinapathy, blindness, and renal failure. Because the pharmacokinetics of absorption of wild-type human insulin or human insulin analogues—when formulated at strengths greater than U-100—is often too slow, too prolonged and too variable relative to the physiological requirements of post-prandial metabolic homeostasis, patients with DM associated with marked insulin resistance often fail to achieve optimal glycemic targets and are thus at increased risk of both immediate and long-term complications. Thus, the safety, efficacy, and real-world convenience of regular and rapid-acting insulin products have been limited by prolongation of PK/PD as the concentration of self-assembled insulin or insulin analogue is made higher than ca. 0.6 mM.

There is a need to preserve zinc-mediated assembly of insulin hexamers but reduce the extent of higher-order hexamer-hexamer self-assembly as a mechanism to achieve a formulation of sufficient chemical stability and of sufficient physical stability to meet or exceed regulatory standards. Chemical degradation refers to changes in the arrangement of atoms in the insulin molecule, such as deamidation of Asn, formation of iso-Asp, and breakage of disulfide bridges. The susceptibility of insulin to chemical degradation is correlated with its thermodynamic stability (as probed by chemical denaturation experiments); because it is the monomer that is the species most susceptible to chemical degradation, its rate is reduced by sequestration of monomers within self-assemblies. Physical degradation refers to fibril formation (fibrillation), which is a non-native form of self-assembly that leads to linear structures containing thousands (or more) of insulin protomers in a beta-sheet rich conformation. Fibrillation is a serious concern in the manufacture, storage and use of insulin and insulin analogues above room temperature. Rates of fibrillation are enhanced with higher temperature, lower pH, agitation, or the presence of urea, guanidine, ethanol co-solvent, or hydrophobic surfaces. Current US drug regulations demand that insulin be discarded if fibrillation occurs at a level of one percent or more. Because fibrillation is enhanced at higher temperatures, patients with DM optimally must keep insulin refrigerated prior to use. Fibrillation of insulin or an insulin analogue can be a particular concern for such patients utilizing an external insulin pump, in which small amounts of insulin or insulin analogue are injected into the patient's body at regular intervals. In such a usage, the insulin or insulin analogue is not kept refrigerated within the pump apparatus, and fibrillation of insulin can result in blockage of the catheter used to inject insulin or insulin analogue into the body, potentially resulting in unpredictable fluctuations in blood glucose levels or even dangerous hyperglycemia. At least one recent report has indicated that insulin lispro (KP-insulin, an analogue in which residues B28 and B29 are interchanged relative to their positions in wild-type human insulin; sold under the trademark Humalog®) may be particularly susceptible to fibrillation and resulting obstruction of insulin pump catheters. Insulin exhibits an increase in degradation rate of 10-fold or more for each 10° C. increment in temperature above 25° C.; accordingly, guidelines call for storage at temperatures<30° C. and preferably with refrigeration. Such formulations typically include a predominance of native insulin self-assemblies.

The present theory of protein fibrillation posits that the mechanism of fibrillation proceeds via a partially folded intermediate state, which in turn aggregates to form an amyloidogenic nucleus. In this theory, it is possible that amino-acid substitutions that stabilize the native state may or may not stabilize the partially folded intermediate state and may or may not increase (or decrease) the free-energy barrier between the native state and the intermediate state. Therefore, the current theory indicates that the tendency of a given amino-acid substitution in the insulin molecule to increase or decrease the risk of fibrillation is highly unpredictable; in particular the lag time observed prior to onset of detectable fibrillation does not correlate with measurements of the thermodynamic stability of the native-state monomer (as probed by chemical denaturation experiments). Whereas a given substitution may stabilize both the overall native state and amyloidogenic partial fold—and so delay the onset of fibrillation—another substitution may stabilize the native state but not the amyloidogenic partial fold and so have little or no effect on the lag time. Still other substitutions may destabilize the native state but stabilize the amyloidogenic partial fold, and so lead to accelerated fibrillation despite its apparent stabilizing properties.

There is a need, therefore for an insulin analogue that displays rapid PK/PD for the treatment of DM under a broad range of insulin concentrations from 0.6 mM to 3.0 mM (typically corresponding to formulation strengths in a range from U-100 to U-500) while exhibiting at least a portion of the activity of the corresponding wild-type insulin, maintaining at least a portion of its chemical and/or physical stability.

### SUMMARY OF THE INVENTION

It is, therefore, an aspect of the present invention to provide insulin analogues that provide zinc-stabilized insulin hexamers of sufficient chemical stability and physical stability to enable their formulation at a range of protein concentrations and in a form that confers rapid absorption following subcutaneous injection. The present invention addresses previous limitations for ultra-concentrated insulin formulations and insulin analogues formulations, namely, that they still do not act sufficiently quickly to optimize post-prandial glycemic control or enable use in insulin pumps. The set of three glutamic acid residues of the present invention, [Glu^{A8}, Glu^{B31}, Glu^{B32}], may be used in combination with B-chain substitutions known in the are to cause accelerated disassembly of insulin hexamers or are associated with more rapid absorption of an insulin analogue following its subcutaneous injection relative to wild-type insulin in a similar formulation.

More particularly, this invention relates to insulin analogues that are modified by the incorporation of (a) Glutamic acid (Glu) at position A8, (b) a two-residue Glu^{B31}-Glu^{B32} extension of the B-chain, and (c) optionally, a non-standard amino acid at position B24. The optional non-standard amino-acid substitution at B24 may be Cyclohexanylalanine or a halogenated derivative of the aromatic ring of Phenylalanine (Phe). Such sequences may optionally contain standard amino-acid substitutions at other sites in the A or B chains of an insulin analogue as known in the art to enhance the rapidity of insulin aborption following subcutaneous injection; examples of the latter are provided by Asp^{B28} (as found in current insulin product sold under the trademark Novolog®) or [Lys^{B28}, Pro^{B29}] (as found in current insulin product sold under the trademark Humalog®).

We sought to circumvent the tendency of insulin hexamers to undergo higher-order self-association at protein concentrations greater than 0.6 mM (standard U-100 formulations). To this end, we sought to place additional negatively charged side chains at the hexamer-hexamer interface as visualized in crystal lattices (FIG. 2). Electrostatic surfaces of the wild-type hexamer are shown in FIGS. 3A and 3B (top and bottom of the hexamer). The analogues of the present invention contain three additional Glutamic acid (Glu) residues as follows. (i) Glu^{B31} and Glu^{B32}. Whereas insulin glargine (the active component of Lantus®) contains additional B-chain residues Arg^{B31} and Arg^{B32} (conferring two additional positive charges), the analogues of the present invention contain acidic residues Glu^{B31} and Glu^{B32} (conferring two additional negative charges). Rather than mediating isoelectric precipitation at neutral pH to form a long-acting depot as sought by Lantus®, this charge reversal reduces the isoelectric point of insulin away from neutrality (pI<5). The predicted electrostatic effects of this acidic extension of the B-chain is shown in FIGS. 3C and 3D. (ii) Glu^{A8}. The principle of electrostatic repulsion is extended by means of stabilizing A-chain substitution Thr^{A8}Glu. The predicted electrostatic effects of Glu^{A8} in concert with the acidic extension of the B-chain is shown in FIGS. 3E and 3F. The negative charges at B31, B32, and A8 are predicted to introduce repulsion between the flat upper and lower surfaces of successive hexamers. Although the present invention is not dependent on or constrained by this theory, the orderly assembly of wild-type insulin hexamers (stacked one atop the other as in crystal lattices) and the electrostatic disruption of such stacking are illustrated in schematic fashion in FIGS. 4A and 4B, respectively. It should be noted also that the acidic B31-B32 tag also attenuates mitogenic cross-binding to the Type 1 IGF receptor (IGF-1R), an effect that is also opposite to the enhanced IGF-1R binding characteristic of insulin glargine.

The three negative charges of Glu^{B31}, Glu^{B32}, and Glu^{A8} (employed in concert in insulin analogues of the present invention) may be combined with substitutions known in the art to destabilize the dimer- or trimer-forming surfaces of the insulin hexamer and so confer more rapid absorption of soluble zinc-containing formulations relative to wild-type insulin in the same or similar formulations. Examples of such substitutions are Asp^{B28} (found in insulin aspart, the active component of the insulin product sold under the trademark Novolog®), [Lys^{B28}, Pro^{B29}] (found in insulin lispro, the active component of the insulin product sold under the trademark Humalog®), and [Lys^{A3}, Glu^{B29}] (found in insulin glulisine, the active component of the insulin product sold under the trademark Apidra®). Combination of the set of three glutamic acids (Glu^{B31}, Glu^{B32}, and Glu^{A8}) with other substitutions or modifications is not, however, restricted to B-chain substitutions employed in the latter three products. Indeed, during the past decade specific chemical modifications to the insulin molecule have been described that selectively modify one or another particular property of the protein to facilitate an application of interest. Whereas at the beginning of the recombinant DNA era (1980) wild-type human insulin was envisaged as being optimal for use in diverse therapeutic contexts, the broad clinical use of insulin analogues in the past decade suggests that a suite of non-standard analogues, each tailored to address a specific unmet need, would provide significant medical and societal benefits. Substitution of one natural amino acid at a specific position in a protein by another natural amino acid is well known in the art and is herein designated a standard substitution. Non-standard substitutions in insulin offer the prospect of enhanced stability or accelerated absorption without worsening of PK/PD as a function of insulin analogue concentration in the range 0.6-3.0 mM. The analogues of the present invention in particular include non-standard modification of Phe^{B24}, such as its substitution by Cyclohexanylalanine (Cha) or a halogenated derivative of the aromatic ring of Phe^{B24}.

The claimed invention circumvents previous design restrictions, including those regarding substitution of Phe^{B24}, through the optional incorporation of a non-standard amino-acid substitution at position B24. This is achieved by substitution of an aromatic amino-acid side chain by a halogen-modified aromatic analogue, similar in size and shape to Phenylalanine, where the analogue then maintains at least a portion of biological activity of the corresponding insulin or insulin analogue containing the native aromatic side chain. The non-standard amino-acid side chain (2-F-Phe^{B24}, 2-Cl-Phe^{B24}, or 2-Br-Phe^{B24} at position B24; also designated ortho-monofluoro-Phe^{B24}, ortho-monochloro-Phe^{B24}, ortho-monobromo-Phe^{B24}, respectively) markedly stabilizes the isolated insulin monomer. A similar stabilization of the insulin monomer is conferred by penta-fluoro-PheB24 wherein each of the five ring hydrogen atoms is replaced by a fluorine atom. The non-standard amino-acid side chain (4-F-Phe^{B24}, 4-Cl-Phe^{B24}, or 4-Br-Phe^{B24} at position B24; also designated para-monofluoro-Phe^{B24}, para-monochloro-Phe^{B24}, para-monobromo-Phe^{B24}, respectively) further modulates the rate of hexamer disassembly and so may be included to enhance the rapid-acting properties of the [Glu^{A8}, Glu^{B31}, Glu^{B32}] family of insulin analogues. The non-standard substitution at B24 may also be Cyclohexanylalanine, a non-planar and non-aromatic ring that permits native-like biological activity but hastens the disassembly of zinc insulin hexamers.

The aromatic amino acid Phenylalanine (Phe) is conserved at position B24 among vertebrate insulin sequences. This is one of three phenylalanine residues in insulin (positions B1, B24, and B25). A structurally similar tyrosine is at position B26. The structural environment of Phe^{B24} in an insulin monomer is shown in a ribbon model (FIG. 5A) and in a space-filling model (FIG. 5B). The aromatic ring of Phe^{B24} is believed to pack against (but not within) the hydrophobic core to stabilize the super-secondary structure of the B chain. Phe^{B24} is believed to lie at the classical receptor-binding surface and has been proposed to direct a change in conformation on receptor binding. Phe^{B24} is also believed to pack at the dimer interface of insulin and so at three interfaces of an insulin hexamer. Its structural environment in the insulin monomer differs from its structural environment at these interfaces. In particular, the surrounding volume available to the side chain of Phe^{B24} is larger in the monomer than in the dimer or hexamer.

Aromatic side chains in insulin, as in globular proteins in general, may engage in a variety of hydrophobic and weakly polar interactions, involving not only neighboring aromatic rings but also other sources of positive- or negative electrostatic potential. Examples include main-chain carbonyl- and amide groups in peptide bonds. Hydrophobic packing of aromatic side chains can occur within the core of proteins and at non-polar interfaces between proteins. Such aromatic side chains can be conserved among vertebrate proteins, reflecting their key contributions to structure or function. An example of a natural aromatic amino acid is phenylalanine. Its aromatic ring system contains six carbons arranged as a planar hexagon. Aromaticity is a collective property of the binding arrangement among these six carbons, leading to π electronic orbitals above and below the plane of the ring. These faces exhibit a partial negative electrostatic potential whereas the edge of the ring, containing five C—H moieties, exhibits a partial positive electrostatic potential. This asymmetric distribution of partial charges gives rise to a quadrapole electrostatic moment and may participate in weakly polar interactions with other formal or partial charges in a protein. An additional characteristic feature of an aromatic side chains is its volume. Determinants of this volume include the topographic contours of its five C—H moieties at the edges of the planar ring.

Non-standard modifications of Phe^{B24} include loss of planarity and aromaticity as associated with its substitution by Cyclohexanylalanine (Cha). Other non-standard modifications of Phe^{B24} preserve aromaticity but result in an alteration in its electrostatic properties. Substitution of one or more hydrogen atoms contained within the ring of Phe^{B24} by a halogen atom (fluorine, chlorine, or bromine; Fl, Cl, or Br) cause characteristic changes in dipole and quadrapole electrostatic moments in association with the electronegativity of these halogen atoms. Substitution of one C—H moiety by a C—F, C-Cl, or C—Br moiety, for example, would be expected to preserve its aromaticity but introduced a significant dipole moment in the ring due to the electronegativity of the halogen atom and consequent distortion of the π electronic orbitals above and below the plane of the ring. Whereas the size of the C—F moiety is similar to that of the native C—H moiety (and so could in principle be accommodated in diverse protein environments), its local electronegativity and ring-specific fluorine-induced electrostatic dipole moment could introduce favorable or unfavorable electrostatic interactions with neighboring groups in a protein. Examples of such neighboring groups include, but are not restricted to, CO—NH peptide bond units, lone pair electrons of sulfur atoms in disulfide bridges, side-chain carboxamide functions (Asn and Gln), other aromatic rings (Phe, Tyr, Trp, and His), and the formal positive and negative charges of acidic side chains (Asp and Glu), basic side chains (Lys and Arg), a titratable side chain with potential pKₐ in the range used in insulin formations (His), titratable N- and C-terminal chain termini, bound metal ions (such as Zn²⁺ or Ca²⁺), and protein-bound water molecules.

Further, the [Glu^{A8}, Glu^{B31}, Glu^{B32}] set of substitutions reduces the cross-binding of insulin to the Type-I IGF receptor (IGF-IR) such that the mitogenic properties insulin are not increased. It is another aspect of the present invention that such an analogue may be formulated in zinc-free formulations at pH 7-8 at strengths from U-100 to U-500 with preservation of PK/PD properties similar to, or more rapid and less prolonged than, those of regular formulations of wild-type human insulin at strength U-100.

In general, the present invention provides an insulin analogue comprising a 32-residue B-chain polypeptide that is extended by two Glu residues (Glu^{B31} and Glu^{B32}) in combination with a variant A-chain containing Glu^{A8}. In one example, the B-chain polypeptide also incorporates [Lys^{B28}, Pro^{B29}] to confer added rapid-acting properties; in another embodiment the analog contains not only [Lys^{B28}, Pro^{B29}], but also 2Br-Phe^{B24} at position B24 to augment chemical and physical stability. In another embodiment, the insulin analogue is a mammalian insulin analogue, such as an analogue of human insulin.

In addition or in the alternative, the insulin analogue may contain a non-standard amino-acid substitution at position 29 of the B chain. In one particular example, the non-standard amino acid at B29 is norleucine (Nle). In another particular example, the non-standard amino acid at B29 is ornithine (Orn).

Also provided is a nucleic acid encoding an insulin analogue comprising a 32-residue B-chain polypeptide that contains a two-residue C-terminal extension (Glu^{B31} and Glu^{B32}) or such a nucleic acid that optionally also incorporates a non-standard amino acid at position B24 or B29 or both. In one example, the non-standard amino acid is encoded by a stop codon, such as the nucleic acid sequence TAG. An expression vector may comprise such a nucleic acid and a host cell may contain such an expression vector.

The invention also provides a method of treating a patient. The method comprises administering a physiologically effective amount of an insulin analogue or a physiologically acceptable salt thereof to the patient, wherein the insulin analogue or a physiologically acceptable salt thereof contains a B-chain polypeptide incorporating a two residue extension (Glu^{B31} and Glu^{B32}) and a Glu^{A8} variant A-chain as described above. In one embodiment, the 2Br-Phe (or other non-standard amino acid) in the insulin analogue administered to a patient is located at position B24. In still another embodiment, the insulin analogue is a mammalian insulin analogue, such as an analogue of human insulin.

### BRIEF DESCRIPTION OF THE SEVERAL VIEWS OF THE DRAWINGS

FIG. 1A is a schematic representation of the sequence of human proinsulin (SEQ ID NO: 1) including the A- and B-chains and the connecting region shown with flanking dibasic cleavage sites (filled circles) and C-peptide (open circles).

FIG. 1B is a structural model of proinsulin, consisting of an insulin-like moiety and a disordered connecting peptide (dashed line).

FIG. 1C is a schematic representation of the sequence of human insulin (SEQ ID NOS: 2 and 3) indicating the position of residue B24 in the B-chain.

FIG. 2 provides structural models of the stacking of insulin hexamers in a crystal lattice. (A) Zinc-stabilized T₆ zinc hexamer (side view) contain two axial zinc ions per hexamer (magenta spheres). The A-chain is shown in dark gray, and B-chain in light gray. Although only 3 hexamers are shown, in the crystal lattice continuous stacking of successive hexamers yields a pseudo-infinite column. Such lattice assembly provides a model for successive hexamer-hexamer interactions in solution. (B) Expansion of interface region (box in panel A). (C) Corresponding model based on the wild-type crystal structure showing the predicted positions of Glu^{A4}, Glu^{B31}, and Glu^{B32} at hexamer-hexamer interface.

FIG. 3 provides an illustration of the electrostatic surfaces. (A and B) Electrostatic surface of the wild-type insulin hexamer based on its crystal structure as a zinc hexamer. Red represents negative electrostatic potential, and blue represents positive electrostatic potential. Top and bottom surfaces are shown in panels A and B. (C and D) Predicted electrostatic surface of a variant insulin hexamer containing B-chain extension Glu^{B32} and Glu^{B32} (green sticks). The color code is otherwise as in panel A. Top and bottom surfaces are shown in panels C and D. (E and F) Predicted electrostatic surface of a variant insulin hexamer containing Glu^{A8} (yellow sticks) as well as B-chain extension Glu^{B32} and Glu^{B32} (green sticks). The color code is otherwise as in panel A. Top and bottom surfaces are shown in panels E and F.

FIG. 4 provides a schematic illustration of wild-type hexamer-hexamer self-association and its proposed prevention by electrostatic engineering. (A) Schematic illustration of successive stacking of zinc insulin hexamers (see also ribbon model in FIG. 2). (B) Addition of acidic extension comprising B-chain residues [Glu^{B31}, Glu^{B32}] (red tags; six per hexamer of which one is hidden behind hexamers (gray)) is designed to prevent hexamer-hexamer self-association by means of electrostatic repulsion. This is predicted to lead to a predominance of disaggregated hexamers even in a U-500 formulation. This model is supported by PD studies in a pig model.

FIG. 5A is a ribbon model of an insulin monomer showing aromatic residue of Phe^{B24} in relation to the three disulfide bridges. The adjoining side chains of Leu^{B15} (arrow) and Phe^{B24} are shown. The A- and B chains are otherwise shown in light and dark gray, respectively, and the sulfur atoms of cysteines as circles.

FIG. 5B is a space-filling model of insulin showing the Phe^{B24} side chain within a pocket at the edge of the hydrophobic core.

FIG. 6 is a pair of graphs showing the results of receptor-binding studies of insulin analogues. (Top Panel) Relative activities for the B isoform of the insulin receptor (IR-B) are determined by competitive binding assay in which receptor-bound ¹²⁵I-labeled human insulin is displaced by increasing concentrations of KP-insulin (▪) or its analogues: [Glu^{B31}, Glu^{B32}]-insulin (♦), [Glu^{A8}, Glu^{B31}, Glu^{B32}]-insulin (▴) and 2-Br-Phe^{B24}_[Glu^{A8}, Glu^{B31}, Glu^{B32}]-insulin (▾). (Bottom panel) Relative activities for the Type I IGF receptor (IGF-1R) are determined by competitive binding assay in which receptor-bound ¹²⁵I-labeled IGF-I is displaced by increasing concentrations of KP-insulin (▪) or its analogues: [Glu^{B31}, Glu^{B32}]-insulin (♦), [Glu^{A8}, Glu^{B31}, Glu^{B32}]-insulin (▴) and 2-Br-Phe^{B24}-[Glu^{A8}, Glu^{B31}, Glu^{B32}]-insulin (▾).

FIG. 7 is a series of graphs regarding pharmacodynamic (PD) analysis of wild-type insulin and insulin analogues in the adolescent Yorkshire pig model. Each of FIGS. 7A-7E, show results of comparative PD studies in a given pig; five individual pigs were tested. FIG. 7A provides baseline comparison of Lilly Humulin U-500 R (▪ and black line) versus Lilly Humalog U-100 (▴ and gray line). FIG. 7B provides [Glu^{B31}, Glu^{B32}]-KP-insulin (♦ and gray line; designated “Hexalog”) at a nominal strength of U-500 (3.0 mM) versus control products Lilly Humulin U-500 R (▪ and black line) and Lilly Humalog U-100 (▴ and gray line). Shaded horizontal arrow at right indicates prolonged tail of Lilly Humulin U-500 R. FIG. 7C shows the results of an independent test in a second pig of [Glu^{B31}, Glu^{B32}]-KP-insulin ( and gray line; designated “Hexalog”) at a nominal strength of U-500 (3.0 mM) versus control product Lilly Humulin U-500 R (▪ and black line). FIG. 7D is a graph of the results from another independent test in a third pig of [Glu^{B31}, Glu^{B32}]-KP-insulin (♦ and gray line; designated “Hexalog”) at a nominal strength of U-500 (3.0 mM) versus control product Lilly Humulin U-500 R (▪ and black line). FIG. 7E shows an independent test of 4-Cl-Phe^{B24} derivative of [Glu^{A8}, Glu^{B31}, Glu^{B32}]-KP-insulin (▴ and gray line; designated “Hexalog-Cle”) at a nominal strength of U-500 (3.0 mM) versus control products Lilly Humulin U-500 R (▪ and ; black lines) and Lilly Humalog U-100 (♦ and gray line).

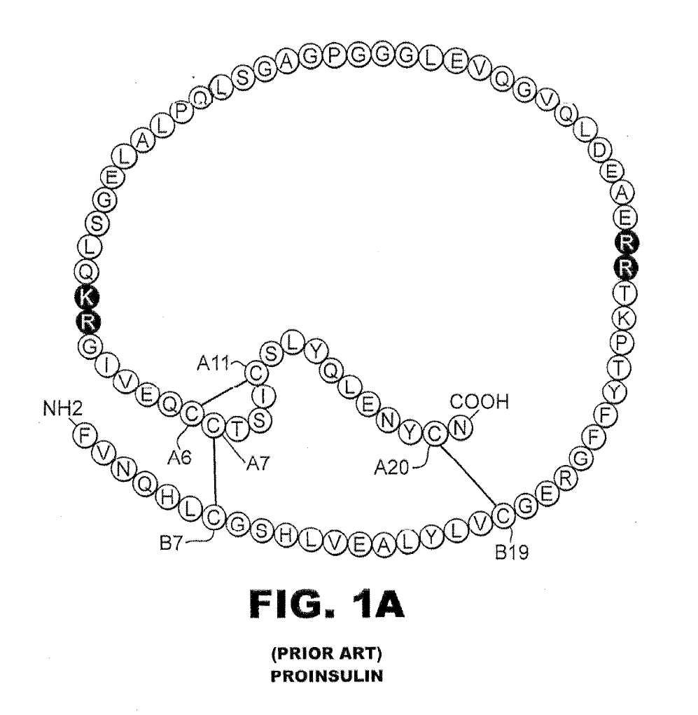

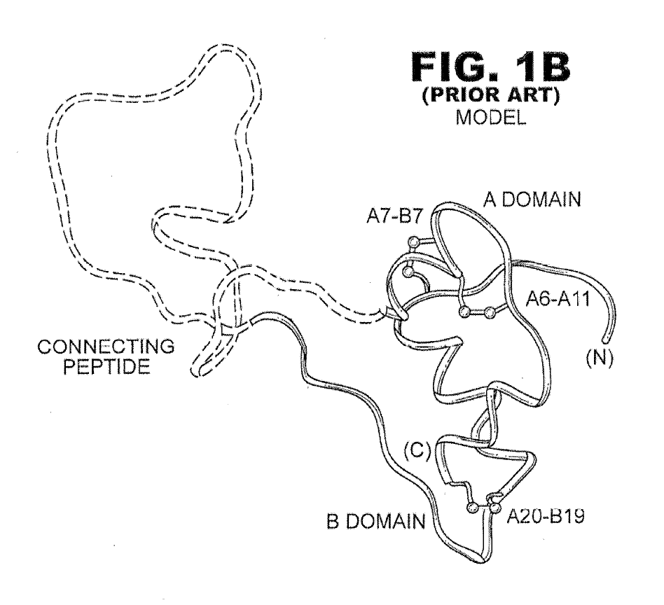

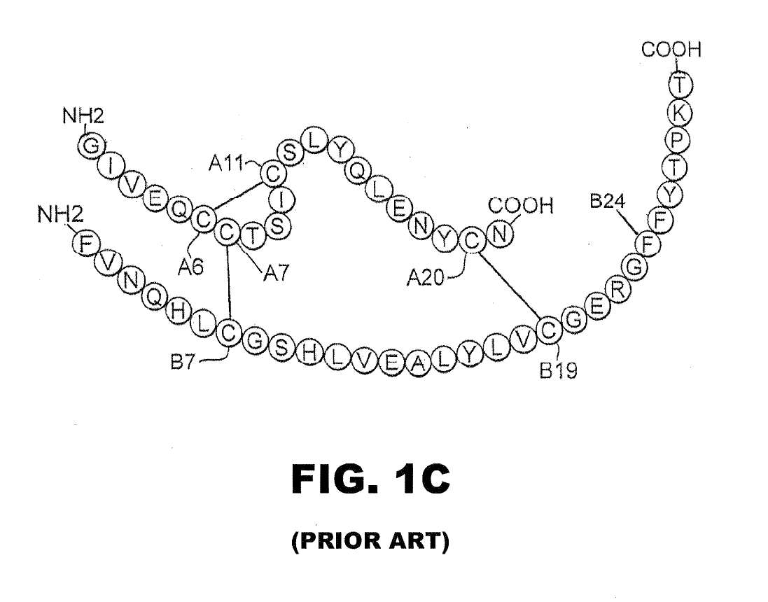

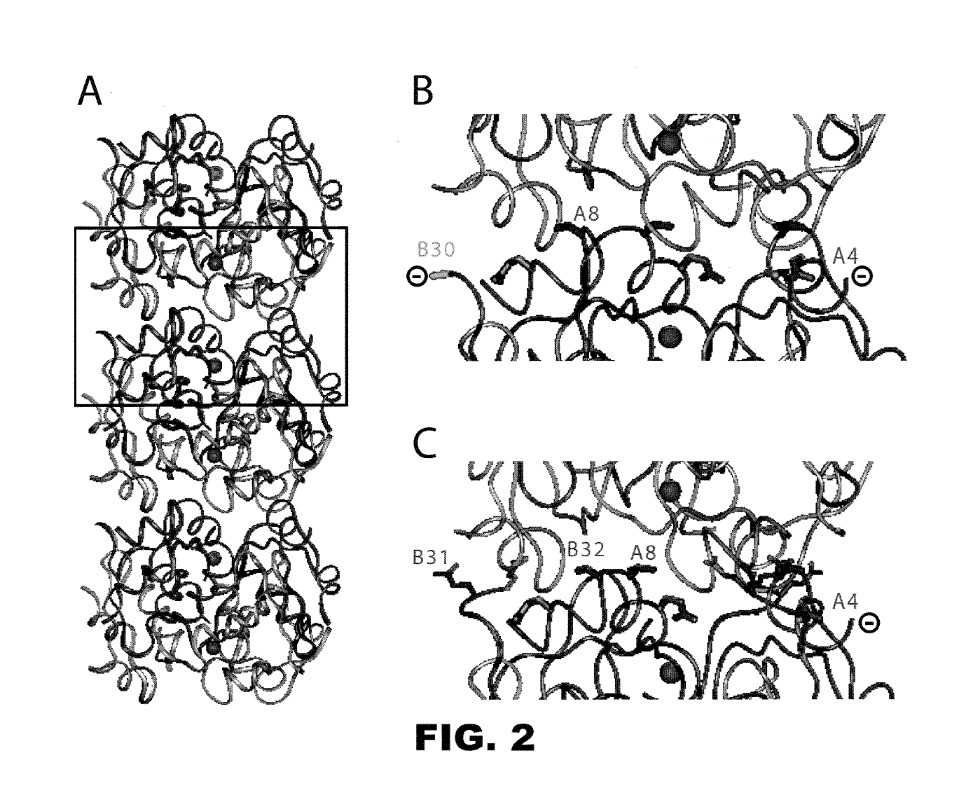

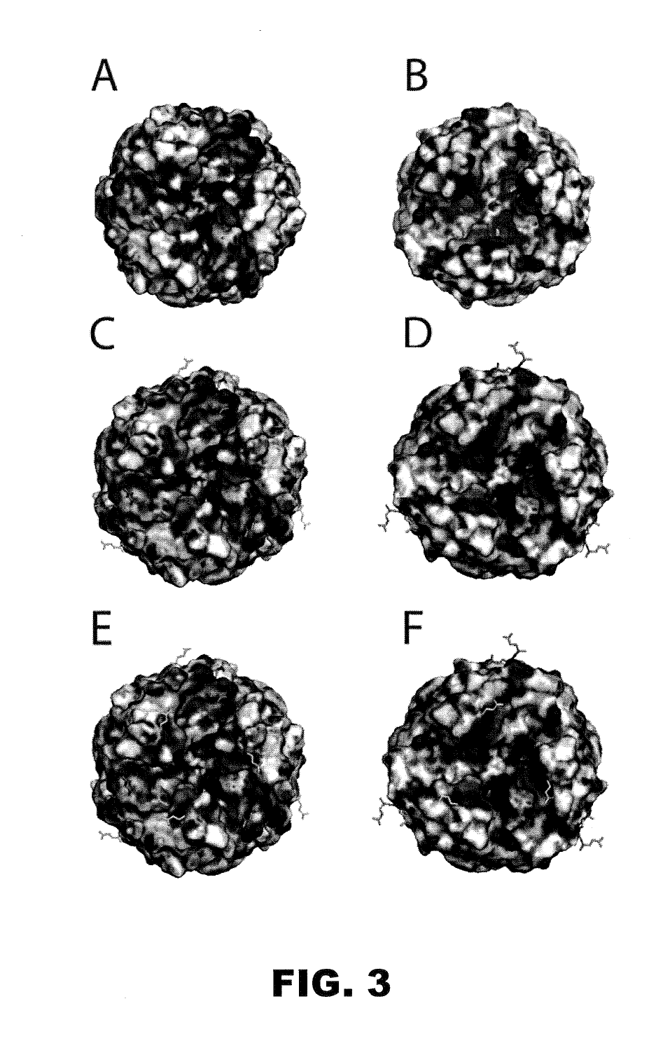

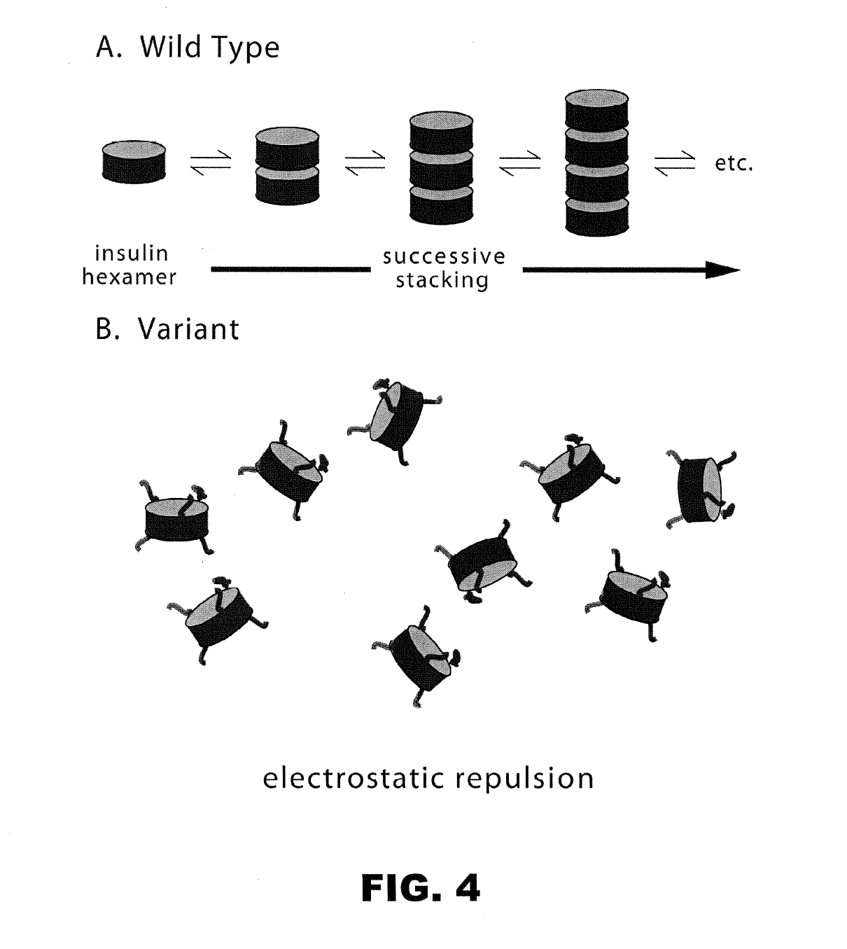

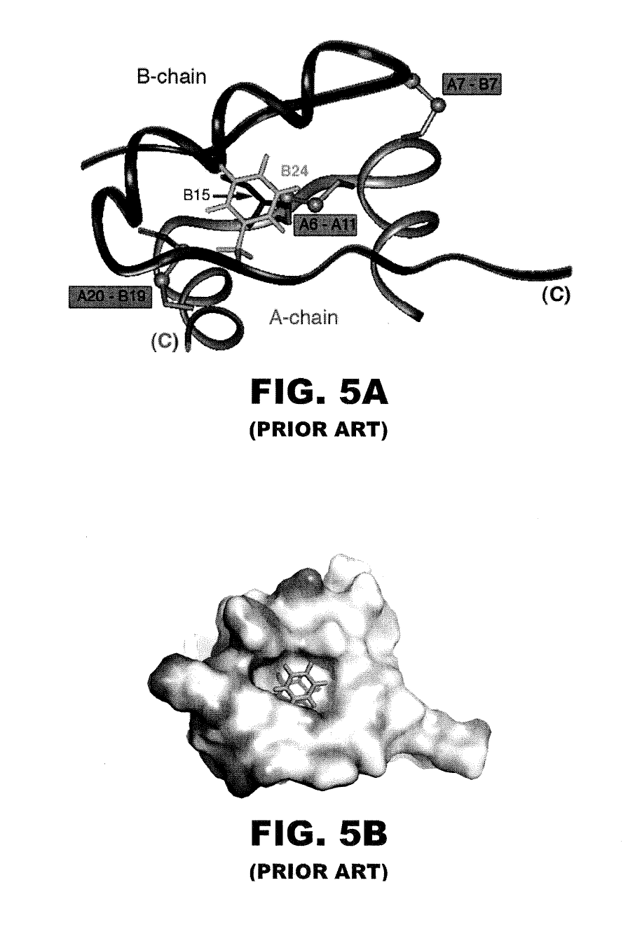

### DETAILED DESCRIPTION OF THE INVENTION

The present invention is directed an insulin analogue that enables rapid PK and PD to be maintained at a broad range of insulin concentrations from U-100 to U-500. The analogue then maintains at least a portion of biological activity of the corresponding unmodified insulin or insulin analogue and maintains similar or enhanced thermodynamic stability and resistance to fibril formation.

The present invention pertains to a set of three Glutamic acid substitutions at positions A8, B31, and B32, optionally in combination with B-chain substitutions known in the art to enhance the rate of absorption of insulin following its subcutaneous injection and optionally in combination with non-standard modification of Phe^{B24}. The latter modifications at B24 include substitution by Cha or by halogen derivatives of the aromatic ring of Phe^{B24} (Fluoro, Chloro, or Bromo). Such modifications are intended to improve the properties of ultra-concentrated insulin formulations with respect to stability or rapidity of absorption following subcutaneous injection. In one instance the insulin analogue contains at least one addition substitution.

Examples are provided by derivatives of insulin lispro ([Lys^{B28}, Pro^{B29}]-insulin; KP-insulin). In either of two embodiments ([Glu^{A8}, Glu^{B31}, Glu^{B32}]-KP-insulin and 2-Br-Phe^{B24}-[Glu^{A8}, Glu^{B31}, Glu^{B32}]-KP-insulin the present invention provides an insulin analogue that exhibits an affinity for the insulin receptor that is similar to those of wild-type insulin or insulin analogues in current clinical use and an affinity for the Type I IGF receptor similar to or lower than that of wild-type human insulin or insulin analogues in current clinical use. The present invention is not limited, however, to the above two derivatives of KP-insulin and its analogues. It is also envisioned that these substitutions may also be made in hexameric analogues derived from animal insulins such as porcine, bovine, equine, and canine insulins, by way of non-limiting examples.

It has been discovered that [Glu^{A8}, Glu^{B31}, Glu^{B32}]-KP-insulin and 2-Br-Phe^{B24}-[Glu^{A8}, Glu^{B31}, Glu^{B32}]-KP-insulin, when formulated in Lilly Diluent and following subcutaneous injection in a male Lewis rat rendered diabetic by streptozotocin, will direct a reduction in blood glucose concentration with a potency similar to or greater that of wild-type human insulin in the same formulation. It has also been discovered that [Glu^{A8}, Glu^{B31}, Glu^{B32}]-KP-insulin and 2-Br-Phe^{B24}-[Glu^{A8}, Glu^{B31}, Glu^{B32}]-KP-insulin, when formulated in a zinc-containing buffer with phenolic preservative and following subcutaneous injection in an anesthetized Yorkshire pig whose endogenous b-cell secretion of insulin was suppressed by intravenous administration of octreotide, will direct a reduction in blood glucose concentration with a potency similar to that of wild-type human insulin in the same formulation and with pharmacokinetics more rapid than those of wild-type insulin at a similar protein concentration and in a similar formulation buffer.

The insulin analogue of the present invention may also contain Asp^{B28} or other substitutions at this site. In addition or in the alternative, the insulin analogue of the present invention may contain a standard or non-standard amino-acid substitution at position 29 of the B chain, which is lysine (Lys) in wild-type insulin. In one particular example, the non-standard amino acid at B29 is norleucine (Nle). In another particular example, the non-standard amino acid at B29 is ornithine (Orn).

Furthermore, in view of the similarity between human and animal insulins, and use in the past of animal insulins in human patients with diabetes mellitus, it is also envisioned that other minor modifications in the sequence of insulin may be introduced, especially those substitutions considered “conservative.” For example, additional substitutions of amino acids may be made within groups of amino acids with similar side chains, without departing from the present invention. These include the neutral hydrophobic amino acids: Alanine (Ala or A), Valine (Val or V), Leucine (Leu or L), Isoleucine (Ile or I), Proline (Pro or P), Tryptophan (Trp or W), Phenylalanine (Phe or F) and Methionine (Met or M). Likewise, the neutral polar amino acids may be substituted for each other within their group of Glycine (Gly or G), Serine(Ser or S), Threonine (Thr or T), Tyrosine (Tyr or Y), Cysteine (Cys or C), Glutamine (Glu or Q), and Asparagine (Asn or N). Basic amino acids are considered to include Lysine (Lys or K), Arginine (Arg or R) and Histidine (His or H). Acidic amino acids are Aspartic acid (Asp or D) and Glutamic acid (Glu or E). Unless noted otherwise or wherever obvious from the context, the amino acids noted herein should be considered to be L-amino acids. Standard amino acids may also be substituted by non-standard amino acids belong to the same chemical class. By way of non-limiting example, the basic side chain Lys may be replaced by basic amino acids of shorter side-chain length (Ornithine, Diaminobutyric acid, or Diaminopropionic acid). Lys may also be replaced by the neutral aliphatic isostere Norleucine (Nle), which may in turn be substituted by analogues containing shorter aliphatic side chains (Aminobutyric acid or Aminopropionic acid).

As used in this specification and the claims, various amino acids in insulin or an insulin analogue may be noted by the amino-acid residue in question, followed by the position of the amino acid, optionally in superscript. The position of the amino acid in question includes the A- or B chain of insulin where the substitution is located. Thus, Phe^{B24} denotes a phenylalanine at the twenty-fourth amino acid of the B chain of insulin.

Although not wishing to be constrained by theory, the present invention envisions that three Glutamic acid residues in combination (Glu^{A8}, Glu^{B31}, and Glu^{B32}) introduces a negative electrostatic potential that has the effect of (i) reducing the extent of hexamer-hexamer interactions in the protein concentration range 0.6-3.0 mM, (ii) enhancing the thermodynamic stability of the insulin analogue, (iii) delaying the onset of fibrillation on gentle agitation above room temperature, and (iv) altering the functional character of the receptor-binding surface so as to decrease cross-binding to the mitogenic Type I IGF receptor. The three Glu residues are not believed to contribute equally to each of these favorable effects. Whereas GluA8 is thought to provide the predominant contribution to the gain in thermodynamic stability, for example, the acidic B-chain extension is believed to make the predominant contribution to the decrease in cross-binding to the IGF receptor. The three Glu residues in concert thus are thought to provide a unique combination of favorable properties.

The analogues of the present invention may optionally contain a non-standard modification of Phe^{B24}. The Phenylalanine at B24 is an invariant amino acid in functional insulin and contains an aromatic side chain. The biological importance of Phe^{B24} in insulin is indicated by a clinical mutation (Ser^{B24}) causing human diabetes mellitus. While not wishing to be bound by theory, Phe^{B24} is believed to pack at the edge of a hydrophobic core at the classical receptor binding surface. The models are based on a crystallographic protomer (2-Zn molecule 1; Protein Databank identifier 4INS). Lying within the C-terminal β-strand of the B chain (residues B24-B28), Phe^{B24} adjoins the central α-helix (residues B9-B19). In the insulin monomer one face and edge of the aromatic ring sit within a shallow pocket defined by Leu^{B15} and Cys^{B19}; the other face and edge are exposed to solvent. This pocket is in part surrounded by main-chain carbonyl and amide groups and so creates a complex and asymmetric electrostatic environment with irregular and loose steric borders. In the insulin dimer, and within each of the three dimer interfaces of the insulin hexamer, the side chain of Phe^{B24} packs within a more tightly contained spatial environment as part of a cluster of eight aromatic rings per dimer interface (Tyr^{B16}, Phe^{B24}, Phe^{B25}, Tyr^{B26} and their dimer-related mates). Irrespective of theory, substitution of the aromatic ring of Phe^{B24} by Cha or halogen derivatives of Phe derivative preserves general hydrophobic packing within the dimer interface while imposing distinct while introducing either a favorable enhancement in the rate of hexamer disassembly or a favorable asymmetric electrostatic interactions within the insulin monomer such that its thermodynamic stability is increased.

The present invention pertains to insulin analogues can be formulated at strengths greater than U-100 and up to U-500 such that, irrespective of the concentration of insulin analogue, the formulation maintains a rapidity of absorption and pharmacologic activity following subcutaneous injection similar to that of a regular wild-type human insulin U-100 formulation; examples of the latter are Humulin® R U-100 (Eli Lilly and Co) or Novalin® R U-100 (Novo-Nordisk). It is envisioned that the substitutions of the present invention may be made in any of a number of existing insulin analogues. For example, the three Glutamic acid residues provided herein may be made in the context of insulin Lispro ([Lys^{B28}, Pro^{B29}]-insulin, herein abbreviated KP-insulin), insulin Aspart (Asp^{B28}-insulin), insulin Glulisine ([LysB³, Glu^{B29}]-insulin), or other modified insulins or insulin analogues, or within various pharmaceutical formulations, such as regular insulin, NPH insulin, lente insulin or ultralente insulin, in addition to human insulin. Insulin Aspart contains an Asp^{B28} substitution and is sold under the trademark Novalog® whereas insulin Lispro contains Lys^{B28} and Pro^{B29} substitutions and is known as and sold under the trademark Humalog®; insulin Glulisine contains substitutions Lys^{B28} and Pro^{B29} and is known as and sold under the trademark Apidra®. These analogues are described in U.S. Pat. Nos. 5,149,777, 5,474,978, and 7,452,860. These analogues are each known as fast-acting insulins.

The amino-acid sequence of human proinsulin is provided, for comparative purposes, as SEQ ID NO: 1.

(human proinsulin)
SEQ ID NO: 1
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Pro-Lys-Thr-Arg-Arg-Glu-Ala-Glu-Asp-
Leu-Gln-Val-Gly-Gln-Val-Glu-Leu-Gly-Gly-Gly-Pro-
Gly-Ala-Gly-Ser-Leu-Gln-Pro-Leu-Ala-Leu-Glu-Gly-
Ser-Leu-Gln-Lys-Arg-Gly-Ile-Val-Glu-Gln-Cys-Cys-
Thr-Ser-Ile-Cys-Ser-Leu-Tyr-Gln-Leu-Glu-Asn-Tyr-
Cys-Asn

The amino-acid sequence of the A chain of human insulin is provided as SEQ ID NO: 2.

(human A chain)
SEQ ID NO: 2
Gly-Ile-Val-Glu-Gln-Cys-Cys-Thr-Ser-Ile-Cys-Ser-
Leu-Tyr-Gln-Leu-Glu-Asn-Tyr-Cys-Asn

The amino-acid sequence of the B chain of human insulin is provided as SEQ ID NO: 3.

(human B chain)
SEQ ID NO: 3
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Pro-Lys-Thr

The amino-acid sequence of the variant A chain of the present invention is provided as SEQ ID. NO. 5.

(variant human A chain)
SEQ ID NO: 5
Gly-Ile-Val-Glu-Gln-Cys-Cys-Glu-Ser-Ile-Cys-Ser-
Leu-Tyr-Gln-Leu-Glu-Asn-Tyr-Cys-Asn

The amino-acid sequence of the extended B chain of human insulin is provided as SEQ ID. NO. 6.

(human B chain)
SEQ ID NO: 6
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Pro-Lys-Thr-Glu-Glu

The amino-acid sequence of the extended B chain of KP-insulin is provided as SEQ ID. NO. 7.

(extended KP B chain)
SEQ ID NO: 7
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Lys-Pro-Thr-Glu-Glu

The amino-acid sequence of the extended B chain of insulin aspart is provided as SEQ ID. NO. 8.

(extended Asp^{B28} B chain)
SEQ ID NO: 8
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Asp-Pro-Thr-Glu-Glu

The amino-acid sequence of the extended B chain of insulin gluline is provided as SEQ ID. NO. 9.

(extended Lys^{A3}, Glu^{B29} B chain)
SEQ ID NO: 9
Phe-Val-Lys-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Pro-Glu-Thr-Glu-Glu

The amino-acid sequence of a B chain of human insulin may be modified with a substitution of a non-standard amino acid at position B24 as described more fully in co-pending International Application No. PCT/US2009/52477, U.S. application Ser. Nos. 12/884,943 and 13/018,011, and U.S. Provisional Patent Application Ser. No. 61/507,324, the disclosures of which are hereby incorporated by reference herein. An example of such a sequence is provided as SEQ. ID. NO 10.

SEQ ID NO: 10
Phe-Val-Xaa₅-Gln-His-Leu-Cys-Gly-Ser-Xaa₄-Leu-Val-
Glu-Ala-Leu-Tyr-Leu-Val-Cys-Gly-Glu-Arg-Gly-Xaa₁-
Phe-Try-Thr-Xaa₂-Xaa₃-Thr-Glu-Glu
[Xaa₁ is Cha, penta-fluoro-Phe, 2-F-Phe, 2-Cl-Phe,
2-Br-Phe, 4-F-Phe, 4-Cl-Phe, 4-Br-Phe; Xaa₂ is
Asp, Pro, Lys, or Arg; Xaa₃ is Lys, Pro, or Ala;
Xaa₄ is His, Asp or Glu; and Xaa₅ is Asn or Lys]

Substitution of a non-standard amino acid at position B24 may optionally be combined with non-standard substitutions at position B29 as provided in SEQ. ID. NO 11.

SEQ ID NO: 11
Phe-Val-Asn-Gln-His-Leu-Cys-Gly-Ser-Xaa₄-Leu-Val-
Glu-Ala-Leu-Tyr-Leu-Val-Cys-Gly-Glu-Arg-Gly-Xaa₁-
Phe-Try-Thr-Xaa₂-Xaa₃-Thr-Glu-Glu
[Xaa₁ is Cha, penta-fluoro-Phe, 2-F-Phe, 2-Cl-Phe,
2-Br-Phe, 4-F-Phe, 4-Cl-Phe, 4-Br-Phe; Xaa₂ is
Asp, Pro, Lys, or Arg; Xaa₂ is Asp, Glu, or Pro;
Xaa₃ is Ornithine, Diaminobutyric acid,
Diaminoproprionic acid, Norleucine, Aminobutric
acid, or Aminoproprionic acid; and Xaa₄ is His,
Asp or Glu]

Trypsin-mediated semisynthesis also employs a synthetic decapeptide containing Glu^{B31} and Glu^{B32} as provided in SEQ ID NO: 12-17.

SEQ ID NO: 12
Gly-Phe-Phe-Tyr-Thr-Pro-Lys-Thr-Glu-Glu.
SEQ ID NO: 13
Gly-Phe-Phe-Tyr-Thr-Lys-Pro-Thr-Glu-Glu.
SEQ ID NO: 14
Gly-Phe-Phe-Tyr-Thr-Asp-Lys-Thr-Glu-Glu.
SEQ ID NO: 15
Gly-Phe-Phe-Tyr-Thr-Pro-Glu-Thr-Glu-Glu.
SEQ ID NO: 16
Gly-Xaa₁-Phe-Tyr-Thr-Asp-Lys-Thr-Glu-Glu.
[Xaa₁ is Cha, penta-fluoro-Phe, 2-F-Phe, 2-Cl-Phe,
2-Br-Phe, 4-F-Phe, 4-Cl-Phe, 4-Br-Phe]
SEQ ID NO: 17
Gly-Xaa₁-Phe-Tyr-Thr-Xaa₂-Pro-Thr-Glu-Glu.
[Xaa₁ is Cha, penta-fluoro-Phe, 2-F-Phe, 2-Cl-Phe,
2-Br-Phe, 4-F-Phe, 4-Cl-Phe, 4-Br-Phe; and Xaa₂ is
Leu, Lys or Asp]

Three analogues of KP-insulin were prepared by trypsin-catalyzed semi-synthesis and purified by high-performance liquid chromatography (Mirmira, R. G., and Tager, H. S., 1989. J. Biol. Chem. 264: 6349-6354.) This protocol employs (i) a synthetic decapeptide representing residues (N)-GF*FYTKPTEE (including modified residue (F*), “KP” substitutions (underlined) and two-residue acidic extension (bold)) and (ii) truncated analogue des-octapeptide[B23-B30]-insulin or Glu^{A8}-des-octapeptide[B23-B30]-insulin. Because the decapeptide differs from the wild-type B23-B30 sequence (GF*FYTPKTEE) by interchange of Pro^{B28} and Lys^{B29} (italics), protection of the lysine &amino group is not required during trypsin treatment. In brief, des-octapeptide (15 mg) and octapeptide (15 mg) were dissolved in a mixture of dimethylacetamide/1,4-butandiol/0.2 M Tris acetate (pH 8) containing 10 mM calcium acetate and 1 mM ethylene diamine tetra-acetic acid (EDTA) (35:35:30, v/v, 0.4 mL). The final pH was adjusted to 7.0 with 10 μL of N-methylmorpholine. The solution was cooled to 12° C., and 1.5 mg of TPCK-trypsin was added and incubated for 2 days at 12° C. An additional 1.5 mg of trypsin was added after 24 hr. The reaction was acidified with 0.1% trifluoroacetic acid and purified by preparative reverse-phase HPLC (C4). Mass spectrometry using matrix-assisted laser desorption/ionization time-of-flight (MALDI-TOF; Applied Biosystems, Foster City, Calif.) in each case gave expected values (not shown). The general protocol for solid-phase synthesis is as described (Merrifield et al., 1982. Biochemistry 21: 5020-5031). 9-fluoren-9-yl-methoxy-carbonyl (F-moc)-protected phenylalanine analogues were purchased from Chem-Impex International (Wood Dale, Ill.).

The above protocol was also employed to prepare the following three insulin analogues: [Glu^{B31}, Glu^{B32}]-KP-insulin, [Glu^{A8}, Glu^{B31}, Glu^{B32}]-KP-insulin, and 2-Br-Phe^{B24}-[Glu^{A8}, Glu^{B31}, Glu^{B32}]-KP-insulin. The insulin analogues were subjected to some or all of the following assays. Biological potency was assessed in a diabetic rat model and by euglycemic clamp in anesthetized Yorkshire pigs; receptor-binding activity values shown are based on ratio of hormone-receptor dissociation constants relative to human insulin (the activity of human insulin is thus 1.0 by definition with standard errors in the activity values otherwise less in general than 25%); thermodynamic stability values (free energies of unfolding; ΔGᵤ) were assessed at 25° C. based on a two-state model as extrapolated to zero denaturant concentration; resistance to fibril formation was evaluated by measurement of lag times (in days) required for initiation of protein fibrillation on gentle agitation at 30° C. in zinc-free phosphate-buffered saline (pH 7.4) as described (Yang, Y., Petkova, A. T., Huang, K., Xu, B., Hua, Q. X., Y, IT, Chu, Y. C., Hu, S. Q., Phillips, N. B., Whittaker, J., Ismail-Beigi, F., Mackin, R. B., Katsoyannis, P. G., Tycko, R., & Weiss, M. A. (2010) An Achilles' Heel in an amyloidogenic protein and its repair. Insulin fibrillation and therapeutic design. J. Biol. Chem. 285, 10806-10821).

Circular dichroism (CD) spectra were obtained at 4° C. and/or 25° C. using an Aviv spectropolarimeter (Weiss et al., Biochemistry 39: 15429-15440). Samples contained ca. 25 μM DKP-insulin or analogues in 50 mM potassium phosphate (pH 7.4); samples were diluted to 5 μM for guanidine-induced denaturation studies at 25° C. To extract free energies of unfolding, denaturation transitions were fitted by non-linear least squares to a two-state model as described by Sosnick et al., Methods Enzymol. 317: 393-409.

The baseline thermodynamic stability of KP-insulin, as inferred from a two-state model of denaturation at 25° C., is 2.8±0.1 kcal/mole. The three analogues exhibited greater stability as follows: [Glu^{B31} Glu^{B32}]-KP-insulin, 3.1±0.1 kcal/mole; [Glu^{A8}, Glu^{B31}, Glu^{B32}]-KP-insulin, 3.6±0.1 kcal/mole; and 2-Br-Phe^{B24}-[Glu^{A8}, Glu^{B31}, Glu^{B32}]-KP-insulin, 4.3±0.1 kcal/mole.

Further, the physical stability of the analogues was found to be markedly greater than that of KP-insulin as evaluated in triplicate during incubation; the proteins were made 300 μM in phosphate-buffered saline (PBS) at pH 7.4 at 45° C. under gentle agitation. The samples were observed for 20 days or until signs of precipitation or frosting of the glass vial were observed. Whereas the lag time for KP-insulin was between 1 and 2 days, the respective lag times of the analogues were prolonged as follows: [Glu^{B31}, Glu^{B32}]-KP-insulin, 5 days; [Glu^{A8}, Glu^{B31}, Glu^{B32}]-KP-insulin, between 12 and 13 days; and 2-Br-Phe^{B24}_[Glu^{A8}, Glu^{B31}, Glu^{B32}]-KP-insulin, not tested.

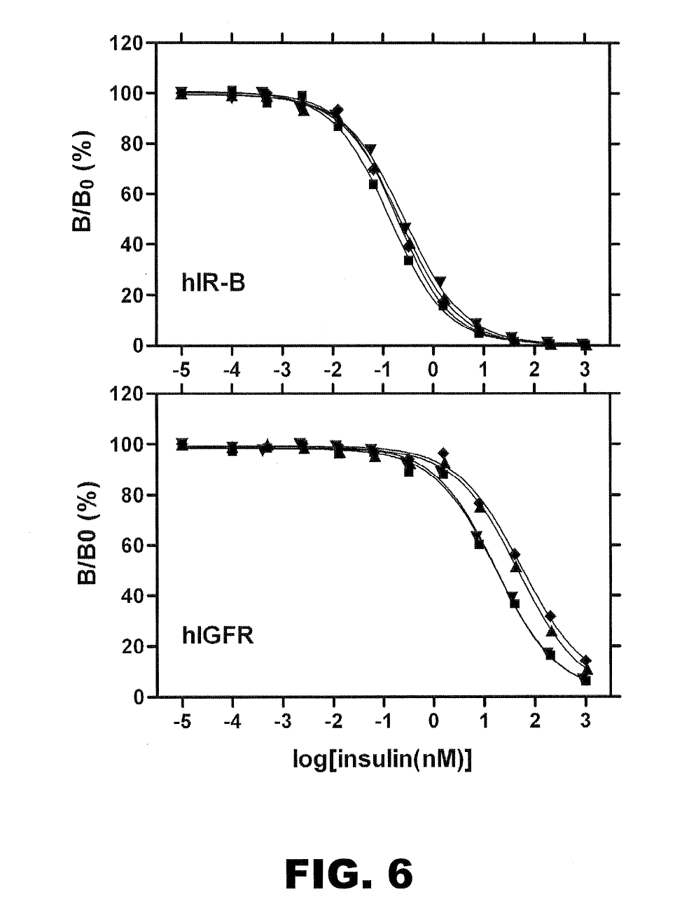

Relative receptor-binding activity is defined as the ratio of the hormone-receptor dissociation constants of analogue to wild-type human insulin, as measured by a competitive displacement assay using ¹²⁵I-human insulin. Microtiter strip plates (Nunc Maxisorb) were incubated overnight at 4° C. with AU5 IgG (100 μl/well of 40 mg/ml in phosphate-buffered saline). Binding data were analyzed by a two-site sequential model. Data were corrected for nonspecific binding (amount of radioactivity remaining membrane associated in the presence of 1 μM human insulin. Corresponding assays were performed using the Type I IGF receptor and ¹²⁵I-labeled human IGF-I as tracer. In all assays the percentage of tracer bound in the absence of competing ligand was less than 15% to avoid ligand-depletion artifacts. The results demonstrated that the affinities of the three analogs are between 45-75% of that of KP-insulin; cross-binding to the IGF receptor is similar to or weaker than that of KP-insulin. Representative binding data are provided in FIG. 6.

To assess hypoglycemic potencies of the insulin analogues, male Lewis rats (mean body mass ˜300 grams) were rendered diabetic by treatment with streptozotocin. (This model provides a probe of potency but not degree of acceleration of pharmacokinetics as (i) wild-type insulin, KP-insulin, and Asp^{B28}-insulin exhibit similar patterns of effects of blood glucose concentration and (ii) these patterns are unaffected by the presence of absence of zinc ions in the formulation at a stoichiometry sufficient to ensure assembly of insulin hexamers.) Protein solutions containing wild-type human insulin, insulin analogues, or buffer alone (protein-free sterile diluent obtained from Eli Lilly and Co.; composed of 16 mg glycerin, 1.6 mg meta-cresol, 0.65 mg phenol, and 3.8 mg sodium phosphate at pH 7.4.) were injected subcutaneously, and resulting changes in blood glucose were monitored by serial measurements using a clinical glucometer (Hypoguard Advance Micro-Draw meter). To ensure uniformity of formulation, insulin analogues were each re-purified by reverse-phase high-performance liquid chromatography (rp-HPLC), dried to powder, dissolved in diluent at the same maximum protein concentration (300 μg/mL) and re-quantitative by analytical C4 rp-HPLC; dilutions were made using the above buffer. Rats were injected subcutaneously at time t=0 with 20 μg insulin in 100 μl of buffer per 300 g rat. This dose corresponds to ca. 67 μg/kg body weight, which corresponds in international units (IU) to 2 IU/kg body weight. Dose-response studies of KP-insulin indicated that at this dose a near-maximal rate of glucose disposal during the first hour following injection was achieved. The rats were randomly selected from a colony of 30 diabetic rats. The two groups exhibited similar mean blood glucose concentrations at the start of the experiment. Blood was obtained from clipped tip of the tail at time 0 and every 10 minutes up to 90 min; in some studies the time period was extended to 180 min or 240 min. The efficacy of insulin action to reduce blood glucose concentration was calculated using the change in concentration over time (using least-mean squares and initial region of linear fall) divided by the concentration of insulin injected. The rat assays done at a dose of 20 micrograms per rat at a protein concentration of 0.6 mM indicated that the three analogues were at least as potent as KP-insulin. In fact, [Glu^{A8} Glu^{B31}, Glu^{B32}] KP-insulin and 2-Br-Phe^{B24}-[Glu^{A8}, Glu^{B31}, Glu^{B32}]-KP-insulin appeared to be more potent than KP-insulin.

To assess PK, PD, and potency of insulin analogues in an animal model predictive of pharmacologic properties in humans, 2F-Phe^{B24} derivatives of Asp^{B10}-containing human insulin analogues were investigated in adolescent Yorkshire farm pigs (weight 35-45 kg). On the day of study, each animal underwent anesthesia induction with Telazol and general anesthesia with isoflurane. Each animal was endotreacheally intubated with continuous monitoring of oxygen saturation and end-tidal expired CO₂. Although the animals were not diabetic, islet function was suppressed in the OR by subcutaneous injection of octreotide acetate (44 mg/kg) approximately 30 min before beginning the clamp study and every 2 h thereafter. After IV catheters were placed and baseline euglycemia established with 10% dextrose infusion, an subcutaneous injection of the insulin was given through the catheter. In order to quantify peripheral insulin-mediated glucose uptake, a variable-rate glucose infusion was given to maintain a blood glucose concentration of approximately 85 mg/dl. This glucose infusion typically will be required for 5-6 hours, i.e., until in control studies of Humulin® glucose infusion rates were typically observed to return to pre-insulin baseline values. Glucose concentrations were measured with a Hemocue 201 portable glucose analyzer every 10 min (with standard error 1.9%).

The computerized protocol for glucose clamping was as described (Matthews, D. R., and Hosker, J. P. (1989) Diabetes Care 12, 156-159). In brief, 2-ml blood samples for insulin assay were obtained according to the following schedule: from 0-40 min after insulin delivery: 5-minute intervals; from 50-140 min: 10-minute intervals, and from 160 min—to the point when GIR is back to baseline: 20-min intervals. For PK/PD a 20-min moving mean curve fit and filter will be applied. PD was measured as time to half-maximal effect (early), time to half-maximal effect (late), time to maximal effect, and area-under-the-curve (AUC) over baseline. For each of these analyses, the fitted curve, not the raw data, were employed in subsequent analyses. Each of three pigs underwent two studies: one with Chlorolog (4-Cl-Phe^{B24}, Lys^{B28}, Pro^{B29} insulin) (and one at the same dosage (0.5 max dose) with U-500 comparator Humulin® R U-500 (Eli Lilly and Co., Indianapolis, Ind.) and U-100 comparators Humalog® and control Humulin® (Lilly Laboratories, Indianapolis, Ind.). The results indicate that the three analogues [Glu^{B31}, Glu^{B32}]-KP-insulin [Glu^{A8}, Glu^{B31}, Glu^{B32}]-KP-insulin, and 2-Br-Phe^{B24}-[Glu^{A8}, Glu^{B31}, Glu^{B32}]-KP-insulin each exhibited potencies at least as high as Humulin® R U-500 and with pharmacodynamics faster than Humulin® R U-500.

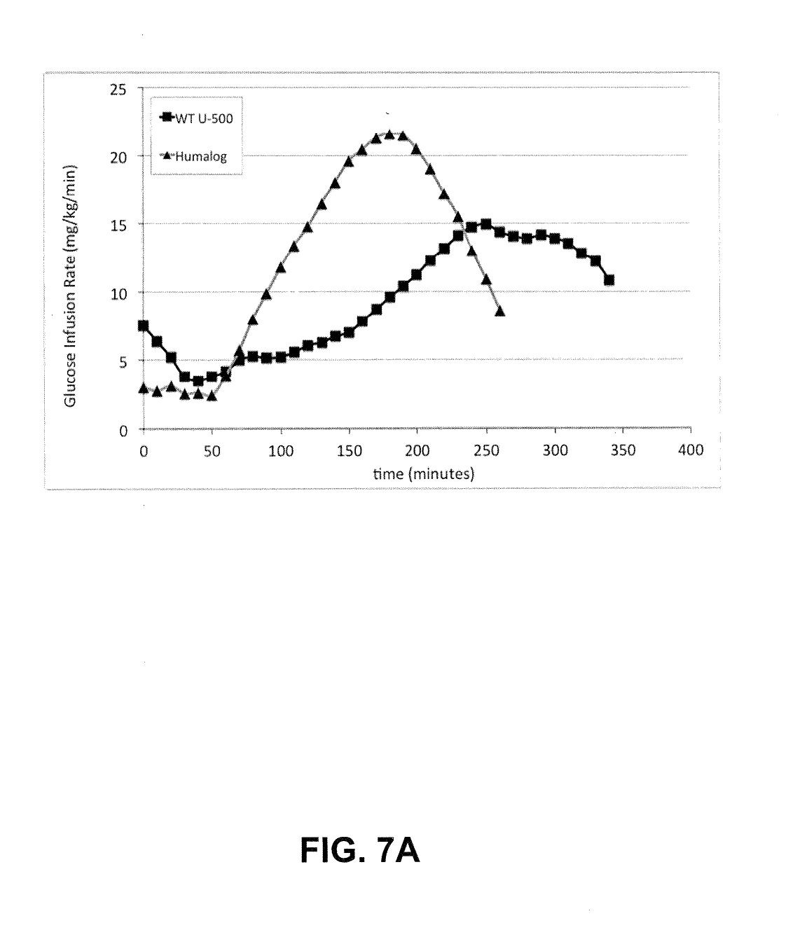

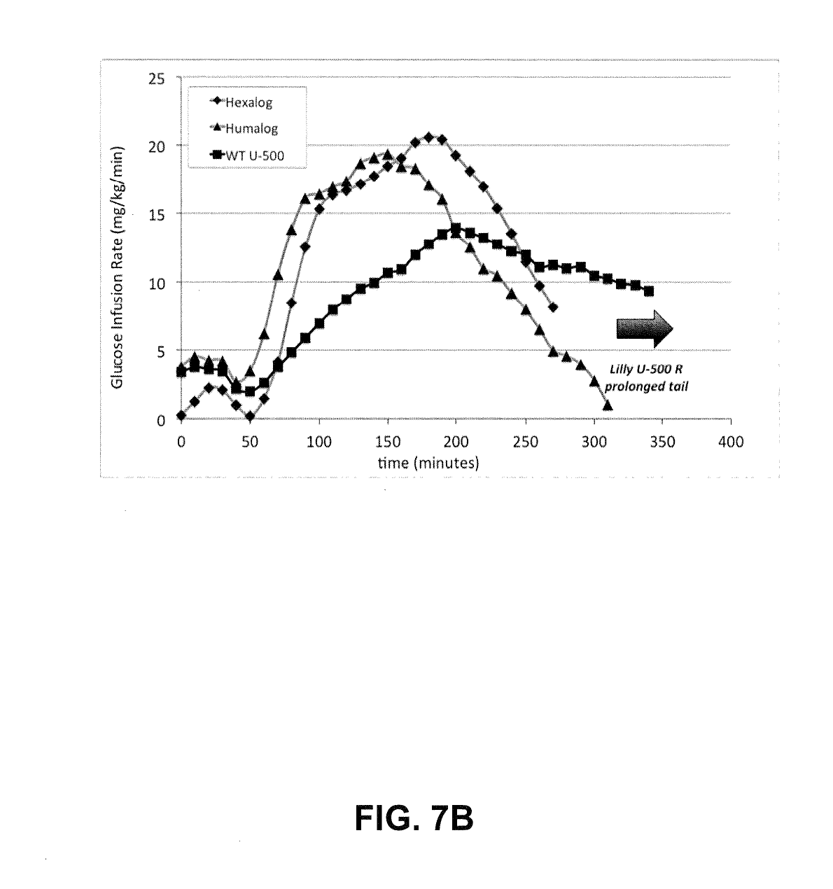

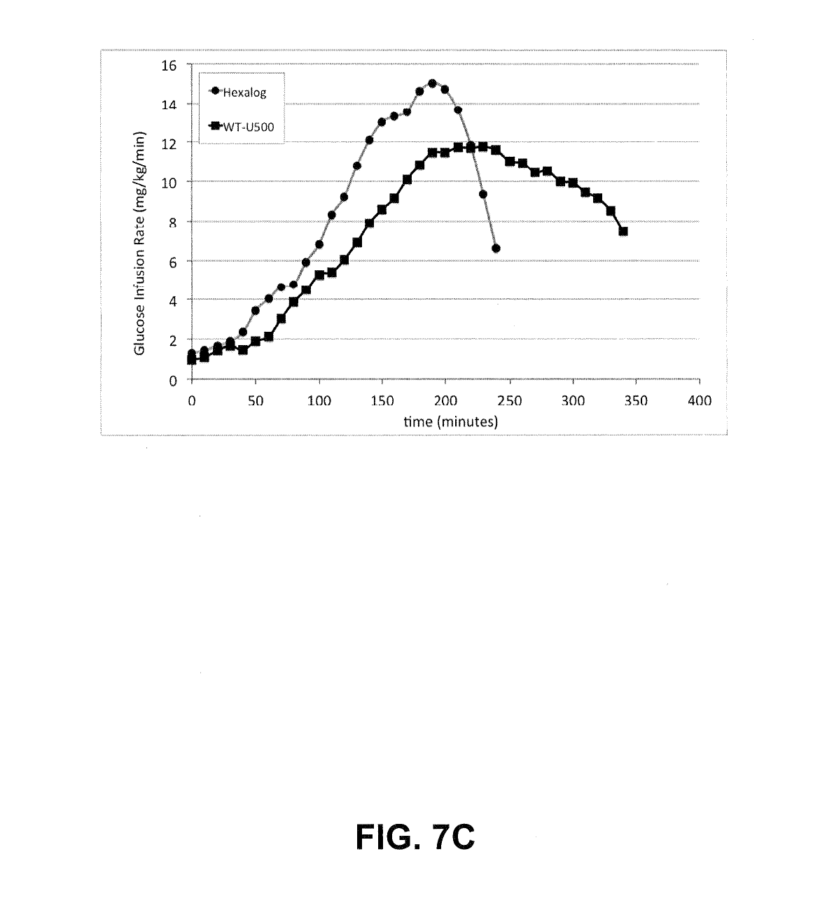

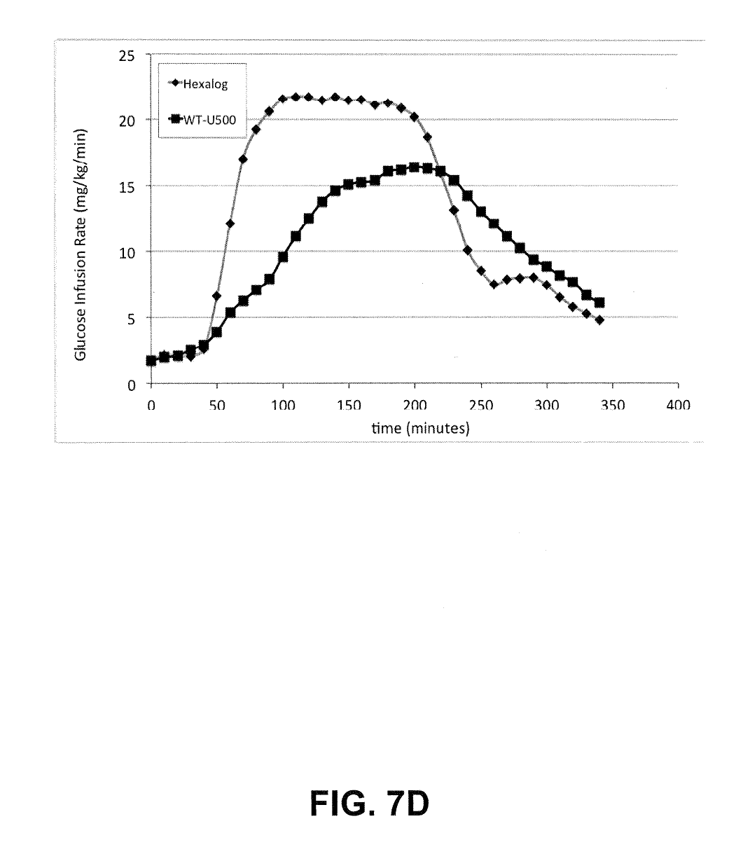

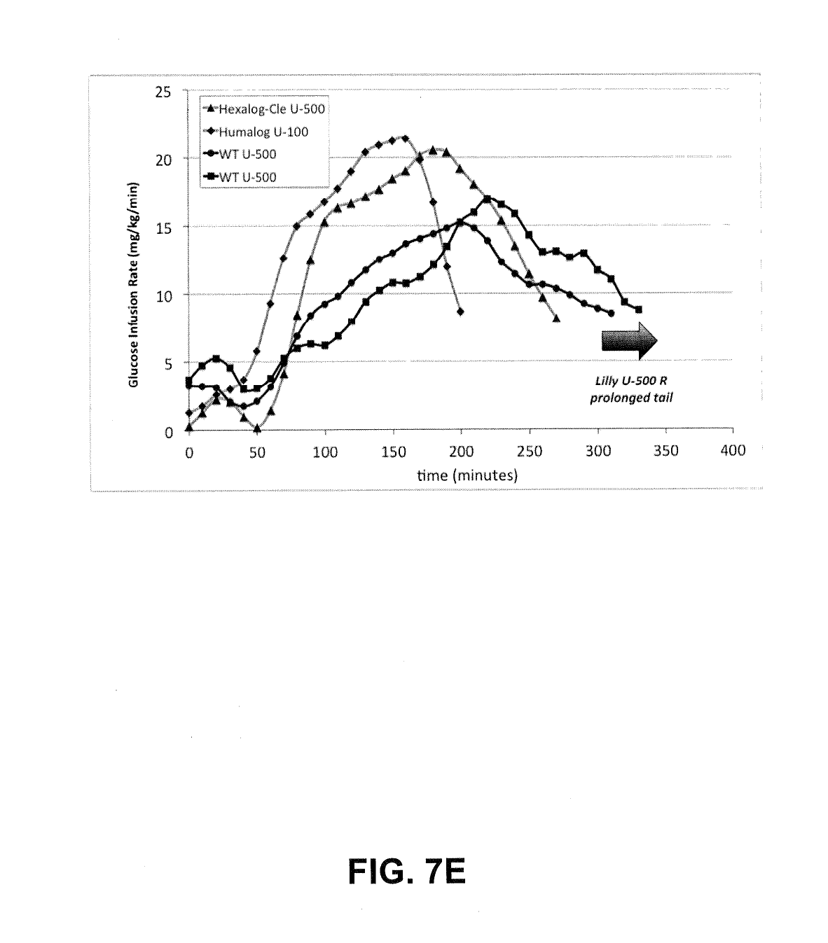

The comparative pharmacodynamics properties of the Glutamic Acid-stabilized insulin analogues were also evaluated as follows with respect to control insulin products manufactured by Eli Lilly & Co: a wild-type regular insulin formulation at a strength of U-500 (Lilly Humulin U-500 R) and prandial insulin analog insulin lispro at a strength of U-100 (Lilly Humalog U-100 R). Because pigs vary in their sensitivity to insulin and with respect to the absorption properties of their skin, comparisons were made within the same pig; a series of independent pigs were thus employed. Data are shown in FIGS. 7A-7E and extracted PD parameters are summarized in Tables 1A-1E. As expected, control studies demonstrated that the PD profile of Lilly Humulin U-500 R was marked prolonged relative to Lilly Humalog U-100 R as illustrated in FIG. 7A. By contrast the PD properties of Lilly Humulin U-500 R were similar to those of insulin lispro when reformulated at a protein concentration of 3.0 mM, i.e., at a strength and in a formulation corresponding to Lilly Humulin U-500 R (data not shown). Such similarity indicates that the lispro modifications do not protect the analogue hexamer from higher-order self-assembly, a finding in accordance with the native-like lattice contacts between such hexamers in the crystal structure of the analog (Ciszak, E., et al. Structure 3, 615-22 (1995)).

TABLE 1A

| | | ½ Tmax | | ½ Tmax |
|---|---|---|---|---|
| | AUC | Early | Tmax | Late |
| | (mg/kg/min²) | (mins) | (mins) | (mins) |
| WT U-500 | 1699 | 152 | 220 | 320 |
| Humalog | 2511 | 73 | 150 | 214 |

Acidic extension of the B-chain by Glu^{B31} and Glu^{B32} in combination with the KP modifications (as known in the art at positions B28 and B29) together yields a novel insulin analogue (designated “Hexalog”) that is rapid acting at very high protein concentrations. FIGS. 7B-7D and Tables 1B-1D demonstrate that the PD properties of [Glu^{B31}, Glu^{B32}]-KP-insulin at a protein concentration of 3.0 mM are markedly faster than Lilly Humulin U-500 R and without a prolonged tail. The areas under the curve suggest that the strength of this formulation is at least U-500.

TABLE 1B

| | | ½ Tmax | | ½ Tmax |
|---|---|---|---|---|
| | AUC | Early | Tmax | Late |
| | (mg/kg/min²) | (mins) | (mins) | (mins) |
| Hexalog U-500 | 2569 | 41 | 120 | 204 |
| Humalog U-100 | 2008 | 48 | 120 | 233 |
| WT U-500 | 2189 | 100 | 180 | 342 |

TABLE 1C

| | | ½ Tmax | | ½ Tmax |
|---|---|---|---|---|
| | AUC | Early | Tmax | Late |
| | (mg/kg/min²) | (mins) | (mins) | (mins) |
| Hexalog U-500 | 1705 | 89 | 170 | 214 |
| WT U-500 | 2293 | 104 | 210 | 330 |

TABLE 1D

| | | ½ Tmax | | ½ Tmax |
|---|---|---|---|---|
| | AUC | Early | Tmax | Late |
| | (mg/kg/min²) | (mins) | (mins) | (mins) |
| Hexalog U-500 | 3812 | 30 | 80 | 204 |
| WT U-500 | 2872 | 77 | 180 | 274 |

FIG. 7E and Table 1E provide analogous data for the modified Glutamic Acid-stabilized insulin analogue in which the 4-Cl-Phe^{B24} modification (i.e., chloro-substitution of the para position of the aromatic ring of Phe^{B24}) accompanies substitutions [Glu^{A8}, Glu^{B31}, Glu^{B32}]-KP-insulin. While not wishing to condition patenability on any particular theory, the 4-Cl-Phe^{B24} modification is believed to further accelerate hexamer disassembly beyond what is effected by the KP modifications at positions B28 and B29. The Glu^{A8} modification is believed to further enhance electrostatic repulsion between hexamers and also to augment the chemical, and physical stability of the monomer, thereby retarding degradation.

TABLE 1E

| | | ½ Tmax | | ½ Tmax |
|---|---|---|---|---|
| | AUC | Early | Tmax | Late |
| | (mg/kg/min²) | (mins) | (mins) | (mins) |
| Hexalog-Cle U-500 | 2,857 | 46 | 140 | 212 |
| Humalog U-100 | 2209 | 38 | 130 | 161 |
| WT U-500 | 2141 | 66 | 170 | 270 |
| WT U-500 | 1667 | 134 | 190 | 281 |

A method for treating a patient comprises administering an insulin analogue containing [Glu^{A8}, Glu^{B31}, Glu^{B32}] modifications or additional amino-acid substitutions in the A or B chain as known in the art or described herein. In still another example, the insulin analogue is administered by an external or implantable insulin pump. An insulin analogue of the present invention may also contain other modifications, such as a tether between the C-terminus of the B chain and the N-terminus of the A chain as described more fully in co-pending U.S. patent application Ser. No. 12/419,169, the disclosure of which is incorporated by reference herein.

A pharmaceutical composition may comprise such insulin analogues and which may optionally include zinc. Zinc ions may be included in such a composition at a level of a molar ratio of between 2.2 and 3.0 per hexamer of the insulin analogue. In such a formulation, the concentration of the insulin analogue would typically be between about 0.1 and about 3 mM; concentrations up to 3 mM may be used in the reservoir of an insulin pump. Modifications of meal-time insulin analogues may be formulated as described for (a) “regular” formulations of Humulin® (Eli Lilly and Co.), Humalog® (Eli Lilly and Co.), Novalin® (Novo-Nordisk), and Novalog® (Novo-Nordisk) and other rapid-acting insulin formulations currently approved for human use, (b) “NPH” formulations of the above and other insulin analogues, and (c) mixtures of such formulations.

Excipients may include glycerol, glycine, arginine, Tris, other buffers and salts, and anti-microbial preservatives such as phenol and meta-cresol; the latter preservatives are known to enhance the stability of the insulin hexamer. Such a pharmaceutical composition may be used to treat a patient having diabetes mellitus or other medical condition by administering a physiologically effective amount of the composition to the patient. The insulin analogues of the present invention may be formulated in the absence of zinc ions and in the presence of 5-10 mM ethylenediaminetetraacetic acid (EDTA) or ethyleneglycoltetraacetic acid (EGTA).

A nucleic acid comprising a sequence that encodes a polypeptide encoding an insulin analogue containing a sequence encoding at least a B chain of insulin with a non-standard amino-acid substitution at position B24 is also envisioned. This can be accomplished through the introduction of a stop codon (such as the amber codon, TAG) at position B24 in conjunction with a suppressor tRNA (an amber suppressor when an amber codon is used) and a corresponding tRNA synthetase, which incorporates a non-standard amino acid into a polypeptide in response to the stop codon, as previously described (Further, 1998, Protein Sci. 7:419-426; Xie et al., 2005, Methods. 36: 227-238). The particular sequence may depend on the preferred codon usage of a species in which the nucleic-acid sequence will be introduced. The nucleic acid may also encode other modifications of wild-type insulin. The nucleic-acid sequence may encode a modified A- or B-chain sequence containing an unrelated substitution or extension elsewhere in the polypeptide or modified proinsulin analogues. The nucleic acid may also be a portion of an expression vector, and that vector may be inserted into a host cell such as a prokaryotic host cell like an E. coli cell line, or a eukaryotic cell line such as S. cereviciae or Pischia pastoris strain or cell line.

For example, it is envisioned that synthetic genes may be synthesized to direct the expression of a B-chain polypeptide in yeast Piscia pastoris and other microorganisms. The nucleotide sequence of a B-chain polypeptide utilizing a stop codon at position B24 for the purpose of incorporating a non-standard amino-acid substitution at that position may be either of the following or variants thereof:

(a) with Human Codon Preferences:

(SEQ ID NO: 18)
TTTGTGAACCAACACCTGTGCGGCTCACACCTGGTGGAAGCTCTCTACCT
AGTGTGCGGGGAACGAGGCTAGTTCTACACACCCAAGACCGAAGAA

(b) with Pichia Codon Preferences:

(SEQ ID NO: 19)
TTTGTTAACCAACATTTGTGTGGTTCTCATTTGGTTGAAGCTTTGTACTT
GGTTTGTGGTGAAAGAGGTTAGTTTTACACTCCAAAGACTGAAGAA

Based upon the foregoing disclosure, it should now be apparent that insulin analogues provided will carry out the objects set forth hereinabove. Namely, these insulin analogues, when formulated under a broad range of protein concentrations from 0.6-3.0 mM (typically corresponding to strengths U-100 to U-500 in the cases of wild-type insulin and prandial insulin analogues), will exhibit enhanced rates of absorption from a subcutaneous depot and pharmacologic action in the regulation of blood glucose concentration while maintaining at least a fraction of the biological activity of wild-type insulin. Further, formulations whose rapid-acting pharmacokinetic and pharmacodynamic properties are maintained at concentrations of insulin analogue as high as 3.0 mM (U-500 strength) will provide enhanced utility in the safe and effective treatment of diabetes mellitus in the face of marked insulin resistance. It is, therefore, to be understood that any variations evident fall within the scope of the claimed invention and thus, the selection of specific component elements can be determined without departing from the spirit of the invention herein disclosed and described.

The following literature is cited to demonstrate that the testing and assay methods described herein would be understood by one of ordinary skill in the art.
Furter, R., 1998. Expansion of the genetic code: Site-directed p-fluoro-phenylalanine incorporation in Escherichia coli. Protein Sci. 7:419-426.
Merrifield, R. B., Vizioli, L. D., and Boman, H. G. 1982. Synthesis of the antibacterial peptide cecropin A (1-33). Biochemistry 21: 5020-5031.
Mirmira, R. G., and Tager, H. S. 1989. Role of the phenylalanine B24 side chain in directing insulin interaction with its receptor: Importance of main chain conformation. J. Biol. Chem. 264: 6349-6354.
Sosnick, T. R., Fang, X., and Shelton, V. M. 2000. Application of circular dichroism to study RNA folding transitions. Methods Enzymol. 317: 393-409.
Wang, Z. X. 1995. An exact mathematical expression for describing competitive biding of two different ligands to a protein molecule FEBS Lett. 360: 111-114.
Weiss, M. A., Hua, Q. X., Jia, W., Chu, Y. C., Wang, R. Y., and Katsoyannis, P. G. 2000. Hierarchiacal protein “un-design”: insulin's intrachain disulfide bridge tethers a recognition α-helix. Biochemistry 39: 15429-15440.
Whittaker, J., and Whittaker, L. 2005. Characterization of the functional insulin binding epitopes of the full length insulin receptor. J. Biol. Chem. 280: 20932-20936.
Xie, J. and Schultz, P. G. 2005. An expanding genetic code. Methods. 36: 227-238.

What is claimed is:

1. An insulin molecule comprising an insulin B-chain polypeptide containing a two-residue extension Glu^{B31} and Glu^{B32} and optionally comprising an insulin A-chain containing the substitution Glu^{A8}.

2. The insulin analogue of claim 1, wherein the B-chain polypeptide additionally comprises a substitution at position B28, position B29, or both.

3. The insulin analogue of claim 2, wherein the B-chain polypeptide additionally comprises the substitution Asp^{B28}.

4. The insulin analogue of claim 2, wherein the the B-chain polypeptide additionally comprises the substitutions Lys^{B28} and Pro^{B29}.

5. The insulin analogue of claim 2, wherein the B-chain polypeptide additionally comprises the substitutions Glu^{B29}.

6. The insulin analogue of claim 1, wherein the B-chain polypeptide additionally comprises a non-standard B24 substitution.

7. The insulin analogue of claim 1, wherein the B-chain polypeptide additionally comprises a substitution at position B24 selected from the group consisting of Cyclohexanylalanine, penta-fluoro-Phenylalanine, ortho-monofluoro-phenylalanine, ortho-monochloro-phenylalanine, and ortho-monobromo-phenylalanine.

8. The insulin analogue of claim 2, wherein the B-chain polypeptide additionally comprises a non-standard substitution at position B29, selected from the group consisting of Norleucine, Aminobutyric acid, Aminopropionic acid, Ornithine, Diaminobutyric acid, and Diaminopropionic acid.

9. The insulin analogue of claim 1, wherein the analogue is an analogue of a mammalian insulin.

10. The insulin analogue of claim 1, wherein the analogue is an analogue of human insulin.

11. A nucleic acid encoding an insulin analogue according to claim 1.

12. A nucleic acid encoding an insulin analogue according to claim 6, wherein the non-standard amino acid at position 24 is encoded by a stop codon.

13. The nucleic acid of claim 12, wherein the stop codon is the nucleic acid sequence TAG.

14. An expression vector comprising the nucleic acid sequence of claim 11.

15. A host cell transformed with the expression vector of claim 14.

16. A method of lowering the blood sugar level of a patient, the method comprising administering a physiologically effective amount of an insulin analogue or a physiologically acceptable salt thereof to the patient, wherein the insulin analogue or a physiologically acceptable salt thereof contains a B-chain polypeptide incorporating the two-residue extension Glu^{B31}-Glu^{B32}.

17. A method of lowering the blood sugar level of a patient, the method comprising administering to the patient a physiologically effective concentration of an insulin analogue of claim 1, or a physiologically acceptable salt thereof, dissolved in a pharmaceutical formulation containing 0.6-3.0 mM insulin analogue.

18. A method of lowering the blood sugar level of a patient, the method comprising administering to the patient a physiologically effective concentration of an insulin analogue of claim 1, or a physiologically acceptable salt thereof, dissolved in a pharmaceutical formulation containing ethylenediaminetetraacetic acid (EDTA) or ethyleneglycoltetraacetic acid (EGTA) at a concentration in the range 5-10 mM.

19. A method of treating a patient comprising the formulation of claim 18 where the insulin solution is injected subcutaneously by a syringe, pen device, or continuously by a pump.


SEQUENCE LISTING (18 sequences)

SEQ ID NO: 1 - protein, 86 aa, Homo sapiens
FVNQHLCGSHLVEALYLVCGERGFFYTPKTRREAEDLQVGQVELGGGPGAGSLQPLALEGSLQKRGIVEQCCTSICSLYQLENYCN

SEQ ID NO: 2 - protein, 21 aa, Homo sapiens
GIVEQCCTSICSLYQLENYCN

SEQ ID NO: 3 - protein, 30 aa, Homo sapiens
FVNQHLCGSHLVEALYLVCGERGFFYTPKT

SEQ ID NO: 4 - protein, 21 aa, Homo sapiens
GIVEQCCESICSLYQLENYCN

SEQ ID NO: 5 - protein, 32 aa, Homo sapiens
FVNQHLCGSHLVEALYLVCGERGFFYTPKTEE

SEQ ID NO: 6 - protein, 32 aa, Homo sapiens
FVNQHLCGSHLVEALYLVCGERGFFYTKPTEE

SEQ ID NO: 7 - protein, 32 aa, Homo sapiens
FVNQHLCGSHLVEALYLVCGERGFFYTDPTEE

SEQ ID NO: 8 - protein, 32 aa, Homo sapiens
FVKQHLCGSHLVEALYLVCGERGFFYTPETEE

SEQ ID NO: 9 - protein, 31 aa, Homo sapiens
  MISC_FEATURE (3)..(3): Asn or Lys
FVXQHLCGSXLVEALYLVCGERGXFTXXTEE

SEQ ID NO: 10 - protein, 31 aa, Homo sapiens
  MISC_FEATURE (10)..(10): His, Asp or Glu
FVNQHLCGSXLVEALYLVCGERGXFTXXTEE

SEQ ID NO: 11 - protein, 10 aa, Homo sapiens
GFFYTPKTEE

SEQ ID NO: 12 - protein, 10 aa, Homo sapiens
GFFYTKPTEE

SEQ ID NO: 13 - protein, 10 aa, Homo sapiens
GFFYTDKTEE

SEQ ID NO: 14 - protein, 10 aa, Homo sapiens
GFFYTPETEE

SEQ ID NO: 15 - protein, 10 aa, Homo sapiens
  MISC_FEATURE (2)..(2): Cyclohexanylalanine, penta fluoro Phe, ortho-monofluoro-Phe, ortho-monochloro-Phe, ortho-monobromo-Phe, para-monofluoro-Phe, para -monochloro-Phe, para -monobromo-Phe
GXFYTDKTEE

SEQ ID NO: 16 - protein, 10 aa, Homo sapiens
  MISC_FEATURE (2)..(2): Cyclohexanylalanine, penta fluoro Phe, ortho-monofluoro-Phe, ortho-monochloro-Phe, ortho-monobromo-Phe, para-monofluoro-Phe, para -monochloro-Phe, para -monobromo-Phe; and Leu, Lys or Asp
GXFYTXPTEE

SEQ ID NO: 17 - DNA, 96 bp, Homo sapiens
tttgtgaaccaacacctgtgcggctcacacctggtggaagctctctacctagtgtgcggggaacgaggctagttctacacacccaagaccgaagaa

SEQ ID NO: 18 - DNA, 96 bp, Homo sapiens
tttgttaaccaacatttgtgtggttctcatttggttgaagctttgtacttggtttgtggtgaaagaggttagttttacactccaaagactgaagaa